(19) United States
(12) Patent Application Publication
(10) Pub. No.: US 2010/0166861 A1
(43) Pub. Date: Jul. 1, 2010

(54) PHARMACEUTICAL FORMULATIONS OF SEVALAMER, OR SALTS THEREOF, AND COPOVIDONE

(76) Inventor: Kelly Noel Lynch, Arvada, CO (US)

Correspondence Address: SANDOZ INC, 506 CARNEFIE CENTER, PRINCETON, NJ, 08540, US

(21) Appl. No.: 12/344,925

(22) Filed: Dec. 29, 2008

(51) Int. Cl. A61K 9/36; A61K 9/28; A61K 9/30; A61K 31/77; B05D 3/12; A61P 7/00

(52) U.S. Cl. 424/480; 424/474; 424/475; 424/78.38; 427/322

(57) ABSTRACT

The present disclosure provides a pharmaceutical composition for the treatment of hyperphosphatemia in mammals. The composition includes sevelamer and copovidone. The composition is provided in the form of a coated tablet having a compressed core. Also disclosed are methods for the manufacture of such tablets, and methods for treating hyperphosphatemia in mammalian patients using the disclosed compositions.

### TECHNICAL FIELD

The present disclosure relates to the field of pharmaceutical compositions, specifically formulations comprising polymeric substances having a desired therapeutic activity and tablets comprising such compositions.

### BACKGROUND

Hyperphosphatemia is an electrolyte disturbance in which there is an abnormally elevated level of phosphate in the patient's blood.

A number of polymeric materials having useful therapeutic activity have been described for treatment of hyperphosphatemia. Many of these polymeric materials function as non-absorbed ion exchange resins in the digestive tract. Such non-absorbed polymeric materials bind or otherwise sequester a target molecule, such as a phosphate ion, and facilitate its removal from the body via the gastrointestinal tract. An example of such a resin includes sevelamer, an epichlorohydrin cross-linked poly(allylamine), disclosed in U.S. Pat. No. 5,496,545, which is incorporated by reference herein. Sevelamer is useful as a phosphate binder, particularly for removing phosphate from patients suffering from renal failure.

Non-absorbed polymer therapeutics such as sevelamer have traditionally presented a number of formulation challenges as the dosages are generally very large (gram quantities), and the resins tend to be extremely hydrophilic. The most desirable formulation for oral delivery of a therapeutic is a direct compression tablet formulation.

However, not all therapeutics, particularly given the high dose requirements of polymeric ion exchange therapeutics, readily lend themselves to tablet formulation. Even if such materials could be rendered into a tablet, it is generally not possible without the significant addition of other materials which assist in the tableting process. Ideally the tablet should contain as much active ingredient as possible with little else in the way of additional materials such that the tablet is as small as possible and easy to administer to the patient.

In addition, once the polymeric material is compressed into a tablet, the tablet may require a coating for ease of administration to the patient. Core polymeric materials such as sevelamer tend to be very hygroscopic, and thus may swell immediately upon contact with the inside of the mouth. Since most coatings contain water, it was further believed that coating such tablets with a water-based coating would be impossible because the hygroscopic tablets would swell during the coating process. Hence, providing a tablet core comprising a hygroscopic material such that a suitable coating may be used in conjunction with that core is another significant challenge to providing the polymeric active ingredient in tablet form.

Accordingly, there is a need to provide suitable dosage forms for hydrophilic aliphatic amine polymers, such as sevelamer, useful as therapeutic agents, which minimize the overall amount of material administered to the patient, which are easy to administer orally, which are stable upon production and storage, and which also have desired hardness and disintegration characteristics. The present disclosure also provides embodiments of pharmaceutical compositions suitable for the treatment of hyperphosphatemia, and methods thereof.

### SUMMARY

A first embodiment of the present disclosure provides a tablet. The tablet includes a tablet core and a tablet coating applied to the exterior of the core. The core may include from about 80 to about 95 percent by weight sevelamer, selected from the group consisting of sevelamer, sevelamer hydrochloride (“HCl”), sevelamer carbonate, and combinations thereof. The core may further include copovidone in an amount ranging from about 1 to about 20 weight percent of the total weight of the core. The core may also include from about 0.01 to about 5 weight percent colloidal SiO₂, and from about 0.01 to about 5 weight percent stearic acid.

The tablet may also have a coating, which may include at least one ingredient selected from the group consisting of titanium dioxide, hydroxypropyl cellulose, hydroxypropylmethyl cellulose having a viscosity of about 50 cP, hydroxypropylmethyl cellulose having a viscosity of about 3 cP, poly(ethylene-glycol) (“PEG”), and mixtures thereof.

The finished tablet may have a hardness ranging from about 30 to about 60 SCU, a friability ranging from about 0 percent to about 0.5 percent, and a disintegration time ranging from about 1 to about 10 minutes in an aqueous solution at a pH of about 7.

Another embodiment of the present disclosure provides a stable pharmaceutical composition useful for treating hyperphosphatemia in mammals, in the form of a tablet. The tablet may include a tablet core and a tablet coating applied to the exterior of the core. The core may include from about 80 to about 95 percent by weight sevelamer, selected from the group consisting of sevelamer, sevelamer HCl, sevelamer carbonate, and combinations thereof. The core may further include copovidone in an amount ranging from about 1 to about 20 weight percent of the total weight of the core. The core may also include from about 0.01 to about 5 weight percent colloidal SiO₂, and from about 0.01 to about 5 weight percent stearic acid.

The tablet may also have a coating, which may include at least one ingredient selected from, but not limited to, the group consisting of titanium dioxide, hydroxypropyl cellulose, hydroxypropylmethyl cellulose having a viscosity of about 50 cP, hydroxypropylmethyl cellulose having a viscosity of about 3 cP, PEG, and mixtures thereof.

The finished tablet may have a hardness ranging from about 30 to about 60 SCU, a friability ranging from about 0 percent to about 0.5 percent, and a disintegration time ranging from about 1 to about 10 minutes in an aqueous solution at a pH of about 7.

A further embodiment of the present disclosure provides a method of making a stable pharmaceutical composition useful for treating hyperphosphatemia in mammals. The method includes a step a) of providing a tablet core mixture. The core mixture may include from about 80 to about 95 percent by weight sevelamer, selected from the group consisting of sevelamer, sevelamer HCl, sevelamer carbonate, and combinations thereof. The core may further include copovidone in an amount ranging from about 1 to about 20 weight percent of the total weight of the core. The core may also include from about 0.01 to about 5 weight percent colloidal SiO₂, and from about 0.01 to about 5 weight percent stearic acid.

The method also includes a step b) of compressing the tablet core mixture with a force ranging from about 1 to about 20 kilonewtons.

The method further includes a step of c) providing a tablet coating mixture. The tablet coating mixture may include at least one ingredient selected from the group consisting of titanium dioxide, hydroxypropyl cellulose, hydroxypropylmethyl cellulose having a viscosity of about 50 cP, hydroxypropylmethyl cellulose having a viscosity of about 3 cP, PEG, and mixtures thereof.

The method may also include a step d) coating the tablet core with the coating mixture.

The finished tablet may have a hardness ranging from about 30 to about 60 SCU, a friability ranging from about 0 percent to about 0.5 percent, and a disintegration time ranging from about 1 to about 10 minutes in an aqueous solution at a pH of about 7.

In some embodiments, the amount of colloidal SiO₂ in the core may be about equal to the amount of stearic acid in the core.

Yet another embodiment of the present disclosure provides a method of treating hyperphosphatemia in mammals. The method includes administering, to a patient having hyperphosphatemia, a composition as disclosed herein.

Additional objects and advantages of the disclosure will be set forth in part in the description which follows, and/or can be learned by practice of the disclosure. The objects and advantages of the disclosure will be realized and attained by means of the elements and combinations particularly pointed out in the appended claims.

It is to be understood that both the foregoing general description and the following detailed description are exemplary and explanatory only and are not restrictive of the disclosure, as claimed.

### DESCRIPTION OF THE EXEMPLARY EMBODIMENTS

The present disclosure will now be described in the more limited aspects of preferred embodiments thereof, including various examples and illustrations of the formulation and use of the present disclosure. It will be understood that these embodiments are presented solely for the purpose of illustrating the invention and shall not be considered as a limitation upon the scope thereof. Unless otherwise noted, all weights and weight percents given are based on the weight of dry components, or the total weight of the dry components.

The presently disclosed pharmaceutical composition may be in the form of a tablet, and in particular a compressed tablet. The tablet may have a tablet core, which may be a compressed tablet core, and a tablet coating. The tablet core may include the pharmaceutically active ingredient(s) of the composition, while the coating may be pharmaceutically inactive.

The tablet core may include certain pharmaceutically acceptable epichlorohydrin cross-linked poly(allylamine) compounds and/or one or more salts thereof which can function as non-absorbed ion exchange resins, for example, sevelamer, sevelamer HCl, and sevelamer carbonate, among other substances.

For the purposes of this disclosure, the term “sevelamer” is explicitly defined to include sevelamer that may be unprotonated, or a protonated or partially protonated salt. For instance, one suitable example of sevelamer may be protonated, partially protonated, or protonated sevelamer HCl.

Sevelamer is a copolymer of epichlorohydrin and prop-2-en-1-amine, also known as poly(allylamine-co-N,N′-diallyl-1,3-diamino-2-hydroxypropane). Sevelamer is believed to have a structure according Formula I, below, wherein the ratio of (a+b):c is about 9:1, and wherein m is a relatively large number indicative of a polymeric network.

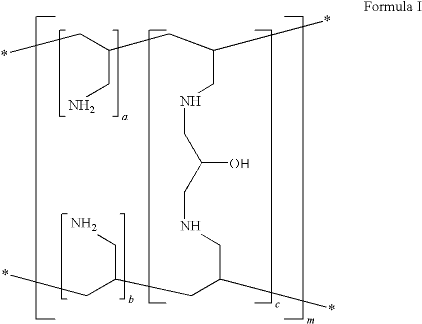

The sevelamer may be present in the composition in an amount ranging from about 80 to about 95 weight percent of the total weight of the tablet core. The sevelamer may be hydrated, containing from about 0 to about 20 weight percent water of the total weight of the sevelamer used.

It is believed that the sevelamer is capable of interacting with and binding phosphate ions, thus sequestering the phosphate for fecal excretion along with the sevelamer, which is not absorbed by the patient. Without desiring to be bound by theory, it is believed that phosphate sequestering and non-absorbent properties of sevelamer are responsible for preventing excess phosphate from being absorbed into the patient's bloodstream. Thus, sevelamer may be particularly useful in treating patients with hyperphosphatemia.

Sevelamer, or a salt thereof, does not readily lend itself to compression into tablet form, and thus the use of other substances such as excipients, binders, and the like may be required during the tableting process.

Accordingly, another important component of the tablet core is a vinylpyrrolidone-vinyl acetate copolymer that is also soluble in water or alcohol. One particularly suitable copolymer is copovidone, also known as copolyvidone, represented by Formula II, below, wherein the ratio of m to n is about 1.2. The mass ratio of the vinylpyrrolidone to the vinyl acetate in the copolymer may be about 6:4.

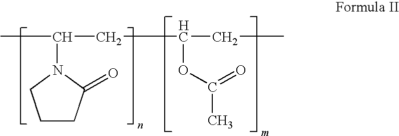

Copovidone may be used as a binding agent, and is believed to provide cohesiveness to the composition during the compression stages of the tableting process. Copovidone is also believed to act as a disintegrant to aid in the breakup and dispersion of the active ingredient after oral consumption of the tablet by a patient. A suitable copovidone composition is available from BASF of Florham Park, N.J., under the tradename KOLLIDON VA64 Fine, although other copovidone preparations may be suitable for the practice of this disclosure.

The copovidone may be present in the tablet core in an amount ranging from about 1 to about 20 weight percent of the total weight of the tablet core, for example from about 5 to about 20, or from about 7 to about 13 percent of the total weight of the tablet core.

Other components that may be present in the tablet core composition may include, but are not limited to, stearic acid and/or colloidal silicon dioxide (SiO₂).

Stearic acid may be used in the pharmaceutical composition as a lubricant or to help prevent the ingredients of the tablet core from clumping together and from sticking during the tableting process. The stearic acid may be present in an amount ranging from about 0.01 to about 5 weight percent of the total weight of the tablet core, for example about from about 0.01 to about 3.6, or from about 0.1 to about 1.0, weight percent of the total weight of the tablet core.

Colloidal silicon dioxide may also be used as a glidant or to promote the flow of powdered ingredients during the tableting process by reducing interparticle friction and cohesion. The colloidal silicon dioxide may be present in an amount ranging from about 0.01 to about 5 weight percent of the total weight of the tablet core, for example about from about 0.01 to about 3.6, or from about 0.1 to about 1.0, weight percent of the total weight of the tablet core.

In some embodiments, the stearic acid and colloidal silicon dioxide may be present in equivalent amounts by weight.

The addition of copovidone, stearic acid, and colloidal SiO₂ to the tablet core unexpectedly and advantageously contributes to achieving the desired properties of hardness and disintegration time for both the sevelamer core and the finished tablet after coating, as described further in the examples below.

The tablet may also have a coating. The coating composition, when dried, may range from about 1 to about 10 weight percent of the total weight of the finished tablet, and more particularly from about 3 to about 7 percent of the total weight of the finished tablet.

The tablet coating may include any or all the following components of Table 1, below, in the ranges given. The ranges indicate the weight percent of the listed coating component, based on the total weight of the coating. The coating composition may be provided in a liquid medium, such as an aqueous or alcoholic solution, mixture, emulsion, or dispersion of the components listed in Table 1.

TABLE 1

| Coating Component | Range (wt %) |
|---|---|
| Titanium Dioxide | 1-50 |
| Hydroxypropyl | 1-50 |
| Cellulose | |
| Hydroxypropylmethyl | 1-50 |
| Cellulose (50 cP) | |
| Hydroxypropylmethyl | 1-50 |
| Cellulose (3 cP) | |
| PEG | 1-50 |

Titanium dioxide may be used as a pigment, for adding color to the tablet coating. The color may enhance the aesthetic appearance of the tablet.

Hydroxypropyl cellulose (“HPC”) may be used as a thickening agent for the tablet coating mixture. HPC may also provide film strength and elasticity to the coating mixture.

Hydroxypropylmethyl cellulose (“HPMC”) may be used as an excipient and controlled-delivery component in oral medicaments. Two different viscosity grades are used in the coating composition, a high viscosity 50 cP grade and a low viscosity 3 cP grade. The mixture of the two different viscosity grades of HPMC is believed to provide film strength and suspension viscosity control to the composition. Without desiring to be bound by theory, it is believed that the ratio of the high viscosity HPMC to the low viscosity HPMC is important in providing a coating having a desired viscosity.

Finally, poly(ethylene-glycol) (“PEG”), is also added to the coating composition as a plasticizer to reduce film brittleness and give flexibility to the coating. The presence of PEG in the coating may aid in the prevention of cracking of the coating.

A tablet coating comprising the above coating composition may be applied to the tablet cores by mixing one or more of the components of Table 1 in a liquid medium, spray coating the tablet cores in a rotating pan, and drying the tablets, as discussed further in the examples below. However, the examples are not meant to limit the method by which the tablets may be coated. Other means of coating the presently disclosed tablet cores with the coating will be readily apparent to those of skill in the art, and need not be described here.

The tablet coatings described herein may protect the tablet core ingredients from deterioration by moisture in the air and may make large or unpleasant-tasting tablets easier to swallow. It is believed that the presently disclosed coating composition may aid in stabilizing the tablet core by providing protection from atmospheric water absorption to the core. The coating may also provide lubrication and glidant properties to the finished tablet.

Some embodiments of the presently disclosed stable pharmaceutical compositions and coated tablets described herein may be manufactured by the following general process.

The powdered ingredients, as described herein (i.e., the sevelamer, copovidone, stearic acid, and colloidal silicon dioxide), may be passed through a mesh screen, such as a 10-30 mesh screen, for example a 20 mesh screen. The ingredients may be combined in a suitable container, such as a bag or a mixer, and mixed until uniformly blended. The blended mix may then be compressed using a tablet press to provide compressed tablet cores.

The coating may be applied to the tablet cores by spray coating the tablets in a rotary pan, or by any other method known to those of skill in the art, which need not be described here.

The compressed, coated tablets may have a hardness ranging from about 30 to about 60 SCU, or more suitably, from about 40 to about 50 SCU. The compressed, coated tablets may also have a friability ranging from about 0 percent to about 0.5 percent, or more suitably, from about 0 percent to about 0.2 percent friability. The finished tablets may also have a disintegration time ranging from about 1 to about 10 minutes, or more particularly from about 2 to about 5 minutes, or even more particularly from about 3 to about 4 minutes in an aqueous solution at a pH of about 7.

In order to demonstrate the advantageous properties of the presently disclosed pharmaceutical compositions, the following non-limiting examples are provided to further illustrate certain embodiments of the present formulations.

### Example 1

Tablet cores for 400 mg sevelamer tablets were prepared in accordance with the following Formulations 1 and 2.

Formulation 1:

| Ingredient | Mg/unit | % w/w |
|---|---|---|
| Sevelamer HCl | 428.000 | 79.99 |
| Copovidone | 103.300 | 19.30 |
| Colloidal SiO₂ | 1.900 | 0.36 |
| Stearic Acid | 1.900 | 0.36 |
| Core Tablet Weight | 535.100 | 100 |

Formulation 2:

| Ingredient | Mg/unit | % w/w |
|---|---|---|
| Sevelamer HCl | 428.000 | 94.00 |
| Copovidone | 24.00 | 5.27 |
| Colloidal SiO₂ | 1.650 | 0.36 |
| Stearic Acid | 1.650 | 0.36 |
| Core Tablet Weight | 455.300 | 100 |

For Formulations 1 and 2, the sevelamer and the copovidone, both in powdered forms, were combined in a polyethylene bag and mixed until substantially uniform, to provide a pre-mix. The pre-mix was then screened through a 20 mesh screen. The colloidal SiO₂ and the stearic acid were also each screened through a 20 mesh screen and then added to the pre-mix to provide a core mixture. The core mixture was mixed until substantially uniform.

The core mixture was then compressed with 0.3000×0.5850 inch tooling using a 16 station Manesty Beta press, using a force ranging from about 1 to about 20 kilonewtons.

Friability is measured by weighing a sample of tablets, loading the tablets into a rotary drum, rotating the drum for about a hundred revolutions, and then weighing the sample of tablets again. The percentage difference before and after the test is the friability of the tablets, expressed as a percent.

Disintegration time is measured by submerging the tablets in an aqueous medium or solution at a pH of about 7. The tablets are held underwater by a disk or disks which prevent said tablets from floating to the top of the medium. The time it takes for the tablets to disintegrate is then observed and measured in minutes and seconds.

Tablet cores of Formulation 1 had a hardness ranging from about 22 SCU to about 36 SCU, and a friability ranging from about 0% for the 22 SCU cores to about 0% friability for the 36 SCU cores. The disintegration time for the cores of Formulation 1 ranged from about 4 minutes, 50 seconds for the 22 SCU cores to about 5 minutes 24 seconds for the 36 SCU cores.

Tablet cores of Formulation 2 had a hardness ranging from about 13 SCU to about 21 SCU, and a friability ranging from about 0. 1% for the 21 SCU cores to about 0.5% friability for the 13 SCU cores. The disintegration time for the 21 SCU cores of Formulation 2 was about 2 minutes, 19 seconds.

The results of this example indicate that increasing the amount of copovidone produced a harder tablet with lower friability and fewer cracks observed along the side wall of the core.

### Example 2

The properties of various 400 mg sevelamer formulations containing other known excipients were evaluated in comparison to an embodiment of the presently disclosed composition, in accordance with the following Table 2. Formulation A is an embodiment of the present disclosure, while Formulations B-E are comparative formulations.

TABLE 2

| Formulation | Ingredient | mg/unit | % w/w |
|---|---|---|---|
| A | Sevelamer HCl | 428.000 | 89.92 |
| | Copovidone | 44.800 | 9.41 |
| | Colloidal SiO₂ | 1.600 | 0.34 |
| | Stearic Acid | 1.600 | 0.34 |
| | Core Tablet Weight | 476.000 | 100.00 |
| B | Sevelamer HCl | 428.000 | 89.92 |
| | Hydroxypropyl Cellulose | 44.800 | 9.41 |
| | Colloidal SiO₂ | 1.600 | 0.34 |
| | Stearic Acid | 1.600 | 0.34 |
| | Core Tablet Weight | 476.000 | 100.00 |
| C | Sevelamer HCl | 428.000 | 89.92 |
| | Hydroxypropyl Cellulose | 44.800 | 9.41 |
| | (Klucel EXF) | | |
| | Colloidal SiO₂ | 1.600 | 0.34 |
| | Stearic Acid | 1.600 | 0.34 |
| | Core Tablet Weight | 476.000 | 100.00 |
| D | Sevelamer HCl | 428.000 | 89.92 |
| | Agglomerated Isomalt | 44.800 | 9.41 |
| | Colloidal SiO₂ | 1.600 | 0.34 |
| | Stearic Acid | 1.600 | 0.34 |
| | Core Tablet Weight | 476.000 | 100.00 |
| E | Sevelamer HCl | 428.000 | 89.92 |
| | Milled Isomalt | 44.800 | 9.41 |
| | Colloidal SiO₂ | 1.600 | 0.34 |
| | Stearic Acid | 1.600 | 0.34 |
| | Core Tablet Weight | 476.000 | 100.00 |

Approximately 500 g batches of tablet cores for each of Formulations A-E were prepared in accordance with the following procedure. For Formulations A-E, the powdered ingredients were passed through a 20 mesh screen in accordance with Table 3, below. The screened ingredients for each formulation were combined in a polyethylene bag and then mixed until substantially uniform, to provide a pre-mix for each of A-E.

TABLE 3

| Ingredient | A | B | C | D | E |
|---|---|---|---|---|---|
| Copovidone | 47 g | n/a | n/a | n/a | n/a |
| (Kollidon | | | | | |
| VA64 Fine) | | | | | |
| Hydroxypropyl | n/a | 47 g | n/a | n/a | n/a |
| Cellulose | | | | | |
| Hydroxypropyl | n/a | n/a | 47 g | n/a | n/a |
| Cellulose | | | | | |
| (Klucel EXF) | | | | | |
| Agglomerated | n/a | n/a | n/a | 47 g | n/a |
| Isomalt | | | | | |
| Milled Isomalt | n/a | n/a | n/a | n/a | 47 g |
| Colloidal SiO₂ | 2 g | 2 g | 2 g | 2 g | 2 g |
| Sevelamer HCl | 450 g | 450 g | 450 g | 450 g | 450 g |

Next, for each formulation, 2 g stearic acid were also screened through a 20 mesh screen and then added to the pre-mixes of A-E to provide core mixtures A-E. Each core mixture was then mixed until substantially uniform.

Formulations A-E were then compressed using a tablet press as described in Example 1 above, in accordance with the parameters of Table 4, below.

TABLE 4

| Parameter | A, B, C, D, E |
|---|---|
| Target Weight (g) | 0.476 g |
| Target Hardness (SCU) | TBD |
| Target Thickness (inches) | TBD |
| Tooling Size | 0.3000 × 0.5850 inch |
| Location: Uppers | Plain |
| Location: Lowers | Plain |
| Press Speed | 560 TPM |

Formulations A and C, when compared to comparative Formulations B, D, and E, exhibited improvements in the properties of hardness, friability, and disintegration rate.

For example, Formulation A exhibited a hardness ranging from about 25 SCU to about 50 SCU. No cracks were observable on the core. Formula A also had a friability of about 0% on the 25 SCU tablets, and tablet abrasion was very little to none. The cores of Formulation A also had a disintegration time ranging from about 1 minute, 27 seconds (for 25 SCU cores) to about 3 minutes, 51 seconds (for 50 SCU cores).

Formulation B had a hardness ranging from about 20 SCU to about 40 SCU. No cracks were observable on the core. Despite also having a friability measured at about 0%, tablet abrasion was noted for the 20 SCU tablets. It was also noted that the tablet surface could easily be rubbed away. Cores of Formulation B had a disintegration time ranging from about 1 minute, 45 seconds to about 2 minutes, 12 seconds.

Formulation C had a hardness ranging from about 25 SCU to about 50 SCU. No cracks were observable on the core. Friability was also measured at about 0% for tablets of all hardnesses, yet minor tablet abrasion was noted for the 25 SCU tablets on one side only. Cores of Formulation C had a disintegration time ranging from about 1 minute, 24 seconds (for the 25 SCU cores) to about 4 minutes, 4 seconds (for the 50 SCU cores).

Formulation D had a hardness ranging from about 20 SCU to about 40 SCU. No cracks were observable on the core. Friability was measured to range from about 0% to about 0.8%, and tablet abrasion was noted for the 20 SCU tablets, which was worse on one side. Cores of Formulation D had a disintegration time ranging from about 1 minute, 5 seconds (for the 20 SCU cores) to about 2 minutes, 32 seconds (for the 40 SCU cores).

Formulation E had a hardness ranging from about 17 SCU about 35 SCU. Cracks were observable on the tablet cores. Friability was measured to range from about 0% to about 0.1%, and little to no tablet abrasion was noted for 25 SCU tablet cores. Cores of Formulation E had a disintegration time ranging from about 1 minute, 2 seconds (for the 25 SCU cores) to about 3 minutes, 02 seconds (for the 35 SCU cores). The 17 SCU cores of Formulation E exhibited complete lamination and severe sticking, extremely undesirable properties.

The results from Formulations A-E indicate that the compressibility of the tablet cores using Klucel EXF and Kollidon VA64 Fine was excellent (Formulations A and C). The tablets did not appear mottled on one side as with other formulations. Disintegration times were acceptable even at 50 SCU hardness for Formulations A and C.

Other excipients that were experimented with and found to be less suitable for core formulation included a microcrystalline cellulose/SiO₂ mixture, granular mannitol, dicalcium phosphate, compressible sugar (sucrose), and powdered mannitol.

### Example 3

In addition to the above described 400 mg sevelamer tablet cores, formulations of 800 mg sevelamer tablet cores having different coatings applied thereupon were also prepared and their properties evaluated, in accordance with Table 5, below.

TABLE 5

| Formulation | Ingredient | mg/unit | % w/w |
|---|---|---|---|
| F | Sevelamer HCl | 948.00 | 90.46 |
| | Copovidone | 94.000 | 8.97 |
| | Colloidal SiO₂ | 3.00 | 0.29 |
| | Stearic Acid | 3.00 | 0.29 |
| | Core Tablet Weight | 1048.00 | 100.00 |
| | Opadry White Y-5-7068 | 110.00 | 9.50 |
| | Purified Water USP | n/a | n/a |
| | Total Tablet Weight | 1158.000 | |
| G | Sevelamer HCl | 948.00 | 90.46 |
| | Copovidone | 94.000 | 8.97 |
| | Colloidal SiO₂ | 3.00 | 0.29 |
| | Stearic Acid | 3.00 | 0.29 |
| | Core Tablet Weight | 1048.00 | 100.00 |
| | Opadry White YS-1-7003 | 110.00 | 9.50 |
| | Purified Water USP | n/a | n/a |
| | Total Tablet Weight | 1158.000 | |

The tablet cores for Formulations F and G were prepared by compression as described above for the 400 mg tablet cores.

The coatings for F and G were then prepared by dispersing the coating ingredient in purified water according to Table 6, below.

TABLE 6

| Ingredient | F | G |
|---|---|---|
| Opadry White Y-5-7068 | 332 g | n/a |
| Opadry White YS-1-7003 | n/a | 332 g |
| Purified Water | 1881 mL | 1881 mL |

A 19 inch diameter side vented Thomas coating pan was then set up for coating the cores according to the following parameters listed in Table 7, below.

TABLE 7

| Parameter | Target Value | Recorded F | Recorded G |
|---|---|---|---|
| Inlet Temperature | 70° C. | 68° C. | 65° C. |
| Outlet Temperature | 40° C. | 44° C. | 45° C. |
| CFM | 375 | 376 | 377 |
| Atomization Air | 35 | 35 | 35 |
| Pan Speed | 12 | 12 | 12 |
| Spray Rate | 30 | 30 | 30 |

The tablet cores of formulations F and G were then each loaded into separate coating pans and sprayed with the corresponding coating formulation from Table 6, in accordance with the following parameters, listed in Table 8, below.

TABLE 8

| Formulation G | | | Formulation F | | |
|---|---|---|---|---|---|
| | | Actual | | | Actual |
| | Actual | Coated | | Actual | Coated |
| Target Total | Solution | Total | Target Total | Solution | Total |
| Weight | Sprayed | Weight | Weight | Sprayed | Weight |
| (mg) | (mL) | (mg) | (mg) | (mL) | (mg) |
| 1083 (3%) | 520 | 1074 | 1083 (3%) | 520 | 1093 |
| 1093 (4%) | 682 | 1106 | 1093 (4%) | 682 | 1103 |
| 1104 (5%) | 861 | 1117 | 1104 (5%) | 861 | 1115 |
| 1114 (6%) | 1023 | 1132 | 1114 (6%) | 1023 | 1129 |
| 1125 (7%) | 1202 | 1145 | 1125 (7%) | 1202 | 1142 |

Tablets coated with Formulation G had disintegration times of 6 minutes, 30 seconds for a 5% coating, and 7 minutes, 30 seconds for a 7% coating.

For tablets of Formulation G, film cracks were noted at all tested coating percentages. For tablets of Formulation F, no cracking was noted. Thus, Formulation F provides an improvement in the coating properties of the tablets over Formulation G.

### Example 6

A batch of 800 mg Sevelamer tablets was prepared to provide tablets, with the amount of sevelamer salt in the tablets adjusted to provide a phosphate binding capacity of about 92.5% and a moisture content of about 10.3%. Compressed tablet cores (Formulation H) were prepared in accordance with Table 9, below. Tablet core compression was carried out as described above.

TABLE 9

| Ingredient (Formulation H) | Mg/unit | % w/w |
|---|---|---|
| Sevelamer HCl | 964.000 | 90.60 |
| Copovidone | 94.000 | 8.83 |
| Colloidal SiO₂ | 3.00 | 0.28 |
| Stearic Acid | 3.00 | 0.28 |
| Core Tablet Weight | 1064.000 | 100.00 |

In addition to the tablet cores, coating compositions H and H′ were also prepared, in accordance with Table 10, below. For coating H, separate suspensions were prepared. For coating H′, both Opadry powders were combined in a single suspension.

TABLE 10

| Ingredient | H | | H′ | |
|---|---|---|---|---|
| Opadry White YS-1-7006 | 75 | g | 131 | |
| Purified Water | 550 | mL | n/a | |
| Opadry White Y-5-7068 | 188 | g | 131 | g |
| Purified Water | 1379 | mL | 1921 | mL |

A 19 inch diameter side-vented Thomas coating pan was then set up for coating the cores according to the following parameters listed in Table 11, below.

TABLE 11

| Parameter | Target Value | Recorded H | Recorded H′ |
|---|---|---|---|
| Inlet Temperature | 70° C. | 68° C. | 65° C. |
| Outlet Temperature | 40° C. | 45° C. | 55° C. |
| CFM | 375 | 373 | 372 |
| Atomization Air | 35 | 35 | 35 |
| Pan Speed | 12 | 12 | 12 |
| Spray Rate | 30 | 31 | 32 |

The tablet cores of formulation H were then each loaded into separate coating pans and sprayed with the corresponding coatings H and H′ from Table 10, in accordance with the following parameters, listed in Table 12, below.

TABLE 12

| Coating H | | | Coating H′ | | |
|---|---|---|---|---|---|
| | | Actual | | | Actual |
| | Actual | Coated | | Actual | Coated |
| Target Total | Solution | Total | Target Total | Solution | Total |
| Weight | Sprayed | Weight | Weight | Sprayed | Weight |
| (mg) | (mL) | (mg) | (mg) | (mL) | (mg) |
| 1099 (3%) | 660 | 1103 | 1099 (3%) | 625 | 1098 |
| 1110 (4%) | 833 | 1112 | 1110 (4%) | 833 | 1113 |
| 1120 (5%) | 1042 | 1132 | 1120 (5%) | 1042 | 1124 |
| 1114 (6%) | 1250 | 1141 | 1131 (6%) | 1260 | 1137 |
| 1125 (7%) | 1458 | 1149 | 1142 (7%) | 1458 | 1146 |

For both H and H′, cracks in the film on the tablet were noted after an approximately 3% coating. The tablets also appeared to exhibit some core erosion during the early stages of coating for both formulations. Tablet samples pulled early were tested for hardness. Cores with erosion tested in the mid-40's SCU, while tablets with no erosion tested in the mid-50's SCU.

### Example 7

A 2.550 kg batch of 800 mg sevelamer tablet cores as described herein was coated with a coating according to Table 13, below, and the properties of the coated tablets were evaluated.

TABLE 13

| Ingredient | Quantity |
|---|---|
| Opadry White Y-5-7068 | 263 g |
| Purified Water | 1490 mL |

A 19 inch diameter side vented Thomas coating pan was then set up for coating the cores according to the following parameters listed in Table 14, below.

TABLE 14

| Parameter | Target Value | Recorded Value |
|---|---|---|
| Inlet Temperature | 65° C. | 66° C. |
| Outlet Temperature | 40° C. | 41° C. |
| CFM | 375 | 375 |
| Atomization Air | 35 | 35 |
| Pan Speed | 12 | 12 |
| Spray Rate | 30 | 31 |

Tablet cores, each weighing about 1037 mg, and having a hardness of about 50 SCU were then spray coated with the suspension from Table 13, in accordance with the parameters of Table 14. At each of the coating intervals of Table 15, below, 50 tablets were removed and retained as a sample. The bulk density of the coated tablets was determined to be about 0.52 g/mL.

TABLE 15

| | | Actual Coated |
|---|---|---|
| Target Total | Actual Solution | Total Weight |
| Weight (mg) | Sprayed (mL) | (mg) |
| 1068 (3%) | 510 | 1070 |
| 1078 (4%) | 680 | 1082 |
| 1089 (5%) | 850 | 1095 |
| 1099 (6%) | 1020 | 1110 |
| 1110 (7%) | 1190 | 1121 |

For tablets having a 5% coating, disintegration time was recorded as about 5 minutes, 12 seconds. For tablets having a 7% coating, disintegration time was recorded as about 6 minutes, 5 seconds.

Tablets from each coating interval were then placed inside an oven at about 50° C. having about 75% relative humidity, both in open dishes and sealed 120 cc HDPE bottles. Only the 7% coated tablets did not exhibit film splitting for all tablets. Some tablets from the 5% and 6% coatings also did not exhibit film splitting.

Tablet cores having a hardness of about 50 SCU result in coated tablets that exhibit some mild abrasion, but not excessive. Accordingly, such tablet cores may be particularly suitable for the practice of the present disclosure, especially when coated with a coating according to Example 7.

### Example 8

As a further example of the bulk manufacture of the presently disclosed sevelamer tablet cores, 400 mg and 800 mg sevelamer HCl tablet cores were prepared according to formulations I (400 mg tablet core) and J (800 mg tablet core), listed in Table 16, below. The tablet cores were made by the process disclosed in the above examples.

TABLE 16

| | I | | J | |
|---|---|---|---|---|
| Ingredient | Quantity (kg) | % w/w | Quantity (kg) | % w/w |
| Sevelamer HCL | 73.80 | 90.8 | 147.85 | 90.8 |
| Copovidone | 7.050 | 8.7 | 14.10 | 8.7 |
| Colloidal SiO₂ | 0.225 | 0.3 | 0.450 | 0.3 |
| Stearic Acid | 0.225 | 0.3 | 0.450 | 0.3 |
| Total | 81.30 | 100 | 162.85 | 100 |

The stable pharmaceutical preparations and tablets described herein may be suitable for administration to mammals in the treatment of hyperphosphatemia, particularly in patients suffering from acute renal failure.

At numerous places throughout this specification, reference has been made to a number of U.S. patents. All such cited documents are expressly incorporated in full into this disclosure as if fully set forth herein.

Other embodiments of the present disclosure will be apparent to those skilled in the art from consideration of the specification and practice of the embodiments disclosed herein. As used throughout the specification and claims, “a” and/or “an” may refer to one or more than one. Unless otherwise indicated, all numbers expressing quantities of ingredients, properties such as molecular weight, percent, ratio, reaction conditions, and so forth used in the specification and claims are to be understood as being modified in all instances by the term “about.” Accordingly, unless indicated to the contrary, the numerical parameters set forth in the specification and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. It is intended that the specification and examples be considered as exemplary only, with a true scope and spirit of the invention being indicated by the following claims.

The foregoing embodiments are susceptible to considerable variation in practice. Accordingly, the embodiments are not intended to be limited to the specific exemplifications set forth hereinabove. Rather, the foregoing embodiments are within the spirit and scope of the appended claims, including the equivalents thereof available as a matter of law.

The patentees do not intend to dedicate any disclosed embodiments to the public, and to the extent any disclosed modifications or alterations may not literally fall within the scope of the claims, they are considered to be part hereof under the doctrine of equivalents.

What is claimed is:

1. A tablet comprising a tablet core and a tablet coating applied to the exterior of the core, wherein:
the tablet core comprises:
from about 80 to about 95 percent by weight sevelamer, wherein the sevelamer is selected from the group consisting of sevelamer, sevelamer HCl, sevelamer carbonate, and combinations thereof,
copovidone in an amount ranging from about 1 to about 20 weight percent of the total weight of the core;
stearic acid in an amount ranging from about 0.01 weight percent to about 5 weight percent of the total weight of the tablet core; and
colloidal SiO₂ ranging from about 0.01 weight percent to about 5 weight percent of the total weight of the tablet core; and
the tablet coating comprises at least one ingredient selected from the group consisting of titanium dioxide, hydroxypropyl cellulose, hydroxypropylmethyl cellulose having a viscosity of about 50 cP, hydroxypropylmethyl cellulose having a viscosity of about 3 cP, poly(ethylene-glycol), and mixtures thereof,
wherein the coating comprises from about 1 to about 10 weight percent of the tablet and the core comprises from about 90 to about 99 percent of the tablet.

2. The tablet of claim 1, wherein the tablet has a hardness ranging from about 30 SCU to about 60 SCU.

3. The tablet of claim 1, wherein the tablet has a hardness ranging from about 40 SCU to about 50 SCU.

4. The tablet of claim 1, wherein the tablet has a friability ranging from about 0% to about 0.5% friability.

5. The tablet of claim 1, wherein the tablet has a friability ranging from about 0% to about 0.2% friability.

6. The tablet of claim 1, wherein the tablet has a disintegration time about ranging from about 1 minute to about 10 minutes in an aqueous solution at a pH of about 7.

7. The tablet of claim 1, wherein the tablet has a disintegration time about ranging from about 2 minutes to about 5 minutes in an aqueous solution at a pH of about 7.

8. The tablet of claim 1, wherein the tablet has a disintegration time about ranging from about 3 minutes to about 4 minutes in an aqueous solution at a pH of about 7.

9. The tablet of claim 1, comprising colloidal SiO₂ ranging from about 0.01 weight percent to about 3.6 weight percent of the total weight of the tablet core.

10. The tablet of claim 1, comprising colloidal SiO₂ ranging from about 0.1 weight percent to about 1.0 weight percent of the total weight of the tablet core.

11. The tablet of claim 1, further comprising stearic acid ranging from about 0.01 weight percent to about 3.6 weight percent of the total weight of the tablet core.

12. The tablet of claim 1, further comprising stearic acid ranging from about 0.1 weight percent to about 1.0 weight percent of the total weight of the tablet core.

13. A pharmaceutical composition useful for treating hyperphosphatemia in mammals, wherein the composition comprises:
a tablet comprising a tablet core and a tablet coating applied to the exterior of the core, wherein:
the core comprises from about 80 to about 95 percent by weight sevelamer, wherein the sevelamer is selected from the group consisting of sevelamer, sevelamer HCl, sevelamer carbonate, and combinations thereof, and wherein the core further comprises copovidone in an amount ranging from about 1 to about 20 weight percent of the total weight of the core, and wherein the core further comprises stearic acid in an amount ranging from about 0.01 weight percent to about 5 weight percent of the total weight of the tablet core, and wherein the core further comprises colloidal SiO₂ ranging from about 0.01 weight percent to about 5 weight percent of the total weight of the tablet core; and
the coating comprises at least one ingredient selected from the group consisting of titanium dioxide, hydroxypropyl cellulose, hydroxypropylmethyl cellulose having a viscosity of about 50 cP, hydroxypropylmethyl cellulose having a viscosity of about 3 cP, poly(ethylene-glycol), and mixtures thereof, wherein the coating comprises from about 1 to about 10 weight percent of the pharmaceutical composition.

14. The composition of claim 13, wherein the tablet has a hardness ranging from about 30 SCU to about 60 SCU.

15. The composition of claim 13, wherein the tablet core has a friability ranging from about 0% to about 0.5% friability.

16. The composition of claim 13, wherein the tablet has a disintegration time ranging from about 1 minute to about 10 minutes in an aqueous solution at a pH of about 7.

17. The composition of claim 13, further comprising colloidal SiO₂ ranging from about 0.01 weight percent to about 3.6 weight percent of the total weight of the tablet core.

18. The composition of claim 13, further comprising stearic acid ranging from about 0.01 weight percent to about 3.6 weight percent of the total weight of the tablet core.

19. A method of making a pharmaceutical composition comprising a tablet, wherein the method comprises the steps of:
a) providing a tablet core mixture comprising from about 80 to about 95 percent by weight sevelamer, wherein the sevelamer is selected from the group consisting of sevelamer, sevelamer HCl, sevelamer carbonate, and combinations thereof, and wherein the core further comprises copovidone in an amount ranging from about 1 to about 20 weight percent of the total weight of the core, and wherein the core mixture further comprises stearic acid in an amount ranging from about 0.01 weight percent to about 5 weight percent of the total weight of the tablet core, and wherein the core mixture further comprises colloidal SiO₂ ranging from about 0.01 weight percent to about 5 weight percent of the total weight of the tablet core;
b) compressing the tablet core mixture with a force ranging from about 1 to about 20 kilonewtons;
c) providing a tablet coating mixture comprising at least one ingredient selected from the group consisting of titanium dioxide, hydroxypropyl cellulose, hydroxypropylmethyl cellulose having a viscosity of about 50 cP, hydroxypropylmethyl cellulose having a viscosity of about 3 cP, poly(ethylene-glycol), and mixtures thereof, and
d) coating the tablet core with the coating mixture;
wherein the tablet has a hardness of ranging from about 30 SCU to about 60 SCU, and a disintegration time ranging from about 1 minute to 10 minutes in an aqueous solution at a pH of about 7.

20. A method of treating a mammalian patient having hyperphosphatemia, comprising administering, to a patient having hyperphosphatemia, a pharmaceutical composition of claim 13.

21. A method of treating a mammalian patient having hyperphosphatemia, comprising administering, to a patient having hyperphosphatemia, a tablet of claim 1.
